(19) United States
(12) Patent Application Publication
(10) Pub. No.: US 2016/0123856 A1
(43) Pub. Date: May 5, 2016

(54) RNA/PRTEIN/DNA PREFERENTIAL FLUID SAMPLE COLLECTION SYSTEM AND METHODS

(71) Applicant: OASIS DIAGNOSTICS CORPORATION, Vancouver, WA (US)

(72) Inventors: PAUL D SLOWEY, VANCOUVER, WA (US); ROBERT L BUCK, VANCOUVER, WA (US); MARY LAUGHLIN, VANCOUVER, WA (US)

(73) Assignee: OASIS DIAGNOSTICS CORPORATION, VANCOUVER, WA (US)

(21) Appl. No.: 14/897,851

(22) PCT Filed: Jun. 12, 2014
(86) PCT No.: PCT/US14/42203

§ 371 Date: Dec. 11, 2015

Related U.S. Application Data
(60) Provisional application No. 61/834,363, filed on Jun. 12, 2013.

(51) Int. Cl. G01N 1/40; C12N 15/10

(52) U.S. Cl. CPC G01N 1/405; G01N 1/4077; C12N 15/1006; G01N 2001/4088

(57) ABSTRACT

Apparatus and methods are provided to obtain RNA-enhanced and protein-enhanced fluid samples which are stable at ambient temperatures. An apparatus includes a filter element made from a fibrous hydrophilic material which preferentially binds and filters cells, DNA-containing macrostructures, mucins and particulates, but does not preferentially bind RNA, RNA-containing macrostructures or proteins. An apparatus may include a sample collector having an absorbent collection pad which also does not preferentially bind RNA, RNA-containing macrostructures or proteins. A method includes obtaining a sample and passing the sample through an RNA-and-protein-preferential filter element to obtain RNA-enhanced and protein-enhanced samples. The method may include use of a RNA-and-protein-preferential absorbent pad material to collect the sample. DNA-rich samples may be obtained by using DNA elution buffer to release the DNA-containing cells and macrostructures after obtaining the RNA-enhanced and protein-enhanced samples.

This Application claims priority to co-pending U.S. Provisional Patent Application Ser. No. 61/834,363, filed Jun. 12, 2013, the disclosure of which is hereby incorporated by reference.

### FIELD OF THE INVENTION

The present invention relates to fluid sample collection apparatus and methods useful for obtaining RNA-enhanced, protein-enhanced and DNA-enhanced sample volumes from single sample collection.

### BACKGROUND

Saliva and other mucosal body fluids obtained noninvasively (or minimally so) are, more and more, the focus of research to develop diagnostic and health monitoring tools, as well as baseline population research. Noninvasive collection is safer, less intimidating and/or socially less stigmatizing for the patient, easier to obtain multiple contemporaneous samples as well as follow up monitoring, easier to obtain under unsanitary field conditions, and can frequently provide rapid objective results (conducive to automated and remote analysis/diagnosis) at the point of care.

Much current research is focused on RNA, RNA metabolites for transcriptomic analyses and proteins for proteomic analyses, as well as traditional DNA sampling, but current apparatus for collecting and processing saliva/mucosal samples are not conducive to providing samples with enhanced RNA fractions to simplify RNA-based test regimes or purified proteins for proteomic analysis, and to render more reliable test results, as well as providing DNA-enhanced samples by applying additional steps to release bound DNA-rich samples. The apparatus and methods described herein provide reliable, low cost, repeatable and scalable means to achieve these goals.

An unexpected benefit of the apparatus and method was discovered by the inventors. RNA and Total Proteins, although of greater and greater interest for research and potential diagnostic uses, including in saliva and other mucous sources, are not very stable. This limited stability has confined traditional saliva collection and isolation to clinical facilities requiring special equipment and methods, and trained personnel, and generally precludes remote and field use. Stabilization methods for RNA test regimes generally require purification at low temperatures, rapid addition of RNA stabilizer and rapid transfer to extremely low temperature long term storage.

The standard operating procedure (SOP) developed at UCLA by Dr. David Wong, called Direct Saliva Transcriptome Analysis (DSTA), requires collection of whole saliva samples on ice, followed by on-site centrifugation at 4° C. to produce saliva supernatant. Until now DSTA has been considered the industry standard. An RNase inhibitor or protein inhibitor cocktail is added to the supernatant and the sample subjected to centrifugation. The centrifugation step creates layers composed of supernatant, mucin, and cellular pellet. The process for removing the saliva supernatant requires skilled lab technicians to carefully pipette the supernatant without disturbing the layers of mucin and cellular pellet, which would reintroduce the DNA containing materials and contaminants intended to be removed through the cold centrifugation step. DSTA requires ice during collection and -processing prior to transferring the separated saliva supernatant to long-term sample storage at −80° C., Although DSTA was a significant improvement over prior collection and isolation processes, DSTA is labor intensive, requires skilled technicians and sophisticated and power hungry lab equipment such as a refrigerated centrifuge, ultra freezer, and pipettes, which effectively limits saliva sampling to clinical and research institutional sites with trained personnel. This creates substantial barriers to the development of non-invasive point-of-care saliva-based research, testing and diagnostics, and dramatically impacts usefulness of saliva-based diagnostics in remote locations, especially in developing countries. Even in developed countries, such procedures are cost prohibitive in the context of general care and diagnostics, and effectively prohibit self-administered sample collection. Therefore, there is a recognized need for saliva sample collection devices and methods providing stable samples at ambient temperatures.

Prior attempts to develop ambient temperature collection and purification methods have focused on conventional mechanical filtration to remove cells, particles and mucin, followed by addition of an RNA stabilization reagent. Although such methods can be effective at removing DNA-containing cellular structures, these filtration methods also tend to bind and/or filter macrostructures containing RNA, and also reduce total protein fractions rather than just removing mucins (up to 20% of total protein count may be lost using these methods). In fact, the Inventors have found that mechanical filtration using membrane-style filter elements may not be feasible because the mucins tend to gel and clog the filter membranes.

Applicant's system solves this long-felt need by producing RNA-enhanced and protein-enhanced samples which are substantially more stable than whole saliva samples. Applicant's apparatus and methods provide the ability to produce homogenous samples for concurrent transcriptomic and proteomic testing, without the need for specially trained personnel, laboratory equipment (such as clean ice for collection and refrigerated centrifuges), and expensive and dangerous stabilizers at the collection site.

Testing by Applicant's research partners at the University of California at Los Angeles (UCLA), using Applicant's apparatus provided by Applicant for the purpose of comparative testing, confirms that saliva samples collected using Applicant's apparatus and methods provide RNA-based and proteome-based test results of comparable accuracy and reliability compared to the DSTA methods (which is the industry standard), without requirement for the specialized equipment and handling procedures. Purified saliva samples produced using Applicant's apparatus and methods contained comparable levels of RNA, mRNA and total proteins compared to equivalent samples obtained using the DSTA method (described above). Additionally, perhaps more importantly, the samples produced using Applicant's apparatus and methods remained stable at ambient temperatures for an unexpectedly long time. Results for mRNA isolation and purification processes showed that samples obtained using Applicant's apparatus and methods retained adequate concentrations of mRNA for effective testing after 14 days at room temperature, even without addition of RNase Inhibitor to the samples. Applicant's apparatus and methods produced samples achieving similar reductions in viable cell contents (having a fraction of less than 4%) from the whole saliva to that produced using the DSTA SOP, without need for centrifugation. The comparative data shows that Applicant's apparatus and methods produce purified saliva samples showing equivalent reduction in cellular and DNA content and improved ambient stability at ambient temperatures to conventional DSTA methods.

### BRIEF SUMMARY OF THE INVENTION AND ADVANTAGES

In an embodiment, a filter is provided having a housing with an inlet to couple to a saliva sample collector and one or more outlets to couple to sample receivers, and a filter element contained within the housing, the filter element being a fibrous hydrophilic material which binds and mechanically filters particulates, mucins, and DNA-containing macrostructures but does not differentially bind or mechanically filter RNA, RNA-containing macrostructures, and does not substantially reduce Total Protein fractions. In another embodiment, the filter element is made from fibrous polyolefin absorbent pad material. In another embodiment the filter element is made from compacted glass wool. In another embodiment the filter element is made from cellulose fiber. In another embodiment, the filter element has an effective pore size in the range 200 to 1,000 nm (0.2 to 1 um) to ensure they are big enough to not remove macromolecular complexes which contain RNA.

In a further embodiment, the filter is combined with a sample collection device having an absorbent pad to collect a saliva sample and pad compression tube to express the saliva sample from the absorbent pad, the absorbent pad made from a hydrophilic fibrous material which traps DNA-containing macrostructures but does not differentially bind or filter RNA or proteins. In another embodiment, the absorbent pad is made from polyolefin fiber.

A method for obtaining RNA-enhanced and Total Protein-enhanced fluid samples is disclosed, including the steps of: obtaining a fluid sample using a collector; passing the fluid sample through a filter having a filter element, the filter element made from a fibrous hydrophilic material which traps DNA-containing macro structures but does not differentially bind or filter RNA, RNA-containing macro structures, or non-mucin proteins within the fluid sample; receiving the filtered RNA-enhanced and Total Protein-enhanced sample in one or more sample receivers. In a further embodiment, the method includes wherein the filter element is further made from polyolefin, glass wool, or cellulose. In a further embodiment, the step of receiving the filtered RNA-enhanced and Total Protein-enhanced sample further includes simultaneously receiving such sample into at least first and second sample receivers, and the method further includes the steps of adding an RNA stabilizer reagent to the first sample receiver and a protein stabilizer reagent to the second sample receiver. In a further embodiment, the method includes the step of, after obtaining one or more RNA-enhanced and Total Protein-enhanced samples, passing a DNA elution buffer through the filter element and into a sample receiver, to obtain a DNA-enhanced sample. In a further embodiment, the method includes the step of, wherein the DNA elution buffer is first applied to the sample collector and expressed from the sample collector through the filter element.

The apparatus and methods described herein convey several advantages over existing apparatus and methods: (1) ease of use, including providing for remote and/or self-administered sample collection; (2) rapidity of sampling by eliminating need for immediate cooling and centrifugation; (3) one-step collection; (4) eliminating the need for specialized accessory equipment, centrifuge or refrigeration; (5) providing relatively stable samples at ambient temperature; (5) reducing need for specially trained personnel; (6) reliably providing an RNA-enhanced and Total Protein-enhanced sample volumes adequate for research and diagnostic testing; (7) permitting use of less toxic and reactive stabilization reagents; (8) providing a collection pad that can be easily modified and adapted to suit the requirements for testing pediatric, neonatal, disabled, and handicapped patients.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

FIG. 1 shows an exploded view of a first embodiment with a sample collection device and sample receivers.

FIG. 2 shows a side view of a filter of a first embodiment.

FIG. 3 shows a perspective view of a second embodiment.

FIG. 4 shows a side view of a second embodiment.

FIG. 5 shows a cutaway side view of a second embodiment.

FIG. 6 shows another side view of a second embodiment.

FIG. 7 shows a side view of a third embodiment.

### DETAILED DESCRIPTION

Before beginning a detailed description of the subject invention, mention of the following is in order. When appropriate, like reference materials and characters are used to designate identical, corresponding, or similar components in differing figure drawings. The figure drawings associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Throughout this disclosure reference is made to saliva as an exemplary and commonly used medium, but the apparatus and methods are equally useful for other mucosal fluids, including but not limited to nasal, vaginal and cervical specimens, as well as urine, and the invention is intended to encompass such uses as well.

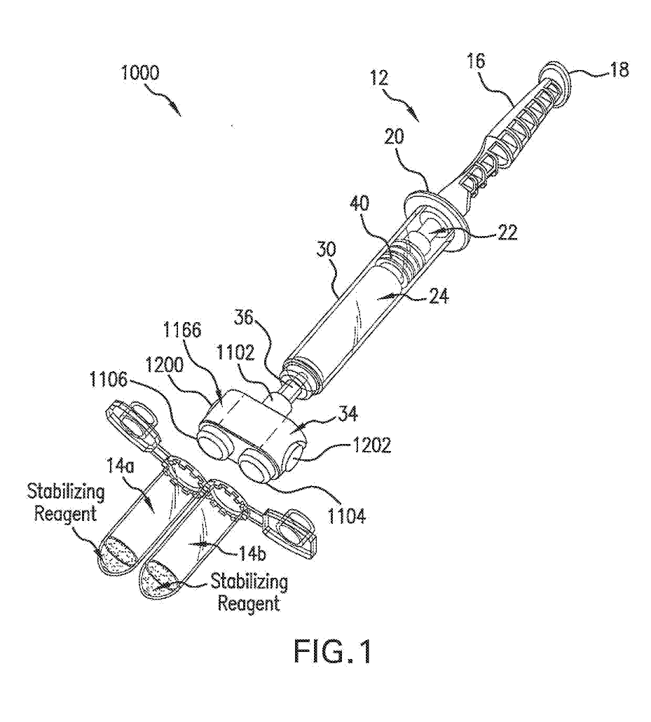

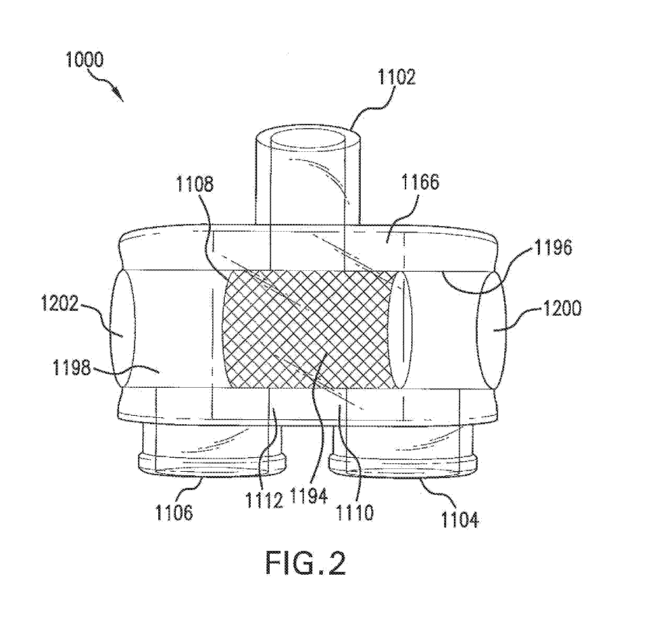

Referring to FIGS. 1-2, a first embodiment includes a fluid sample filter-distributor 1000. Filter-distributor 1000 includes a body 1166 having an inlet port 1102 and first and second outlet ports 1104, 1106, respectively. Filter chamber 1108 is in fluid communication with opposed first and second distribution channels 1110, 1112. Each of first and second distribution channels 1110, 1112, extend from filter chamber 1108, to first and second outlet ports 1104, 1106, respectively.

In the embodiment, filter chamber 1108 is an elongated chamber of constant cross-section extending through body 1166, from a first open end 1196 to a second open end 1198 at opposing sides of body 1166. The cross-section is adapted to snugly receive a desired filter element 1194. In the embodiment, the cross-section is circular. First and second sealing plugs 1200, 1202, are provided to sealingly insert into first and second open ends 1196, 1198, respectively. In the embodiment, filter-distributor 1000 is constructed symmetrically to ensure even sample distribution between outlet ports 1104, 1106.

Dual outlet ports 1104, 1106 are couplable to couplable to sample receivers 14a, 14b. In the embodiment, sample receivers 14a, 14b are pre-loaded with an RNA stabilizing reagent and a proteomic stabilizing reagent, respectively. Alternatively, stabilizing reagents may be added after sample volume collection. Dual outlet ports 1104, 1106, with sample receivers 14a, 14b, provide ability to obtain nonheterogeneous volumes from the same sample collector for concurrent, immediately stabilized RNA and proteomic testing and analyses.

Filter element 1194 is made from a hydrophilic material which preferentially traps cells and other DNA-containing macrostructures but does not preferentially bind RNA or total proteins. In the embodiment, filter element 1194 is made from polyolefin fiber, having a density of 0.077 g/cc. In an alternative embodiment, filter element 1194 is made from glass wool. Glass wool effectively binds DNA-containing macrostructures without preferentially binding RNA or proteins, and is easily compressed to obtain a desired effective pore size while minimizing dead space/void volumes which may retain excessive volumes of sample fluid. In another alternative embodiment, filter element 1194 is made from cellulose.

Cellulose effectively binds mucin without preferentially binding RNA, and is easily compressed to obtain a desired effective pore size. Cellulose, although generally effective in this regard, has certain disadvantages in that it may bind some proteins of interest and retain unrecoverable fluid volume, such that it may not be desirable for certain applications.

In the embodiment, first and second sealing plugs 1200, 1202, have a depth (indicated by “d”) which may be selected to compress filter element 1194 in order to reduce effective pore size and/or to reduce dead space which may retain excessive volumes of sample fluid to ensure adequate filtered volume is collected.

Filter-distributor 1000 is effective to obtain concurrent samples having enhanced RNA and protein fractions. In the embodiment, filter-distributor 1000 is combined with fluid sample collector 12 (to obtain substantially greater efficiency in RNA, mRNA and protein fraction increases. Fluid sample collector 12 includes a handle 16 having opposing first and second ends 18, 20, and a sample sufficiency indicator 22 coupled to handle 16. Handle second end 20 and sufficiency indicator 22 form a cavity adapted to receive sample collection pad 24 having opposing ends 26 and 28, respectively, and a sealing member 40 disposed around the handle end proximate sufficiency indicator 22, which seals against the interior of pad compression tube 30 (acting as a plunger). In the embodiment, sufficiency indicator 22 is a wrap-around light pipe. Sample collection pad 24 is partially contained within and extending from handle second end 20 and in contact with the sufficiency indicator 22. In the embodiment, sample collection pad 24 has a cylindrical cross section. Pad compression tube 30 includes a first open end 32 to go over sample collection pad 24 and second end 20 of handle 16, an opposing second end 34, and an outlet port 36 proximal to pad compression tube second end 34 and in fluid communication with the pad compression tube interior. Fluid sample collector includes internal vents 38 to vent air while expressing sample from sample collector 12.

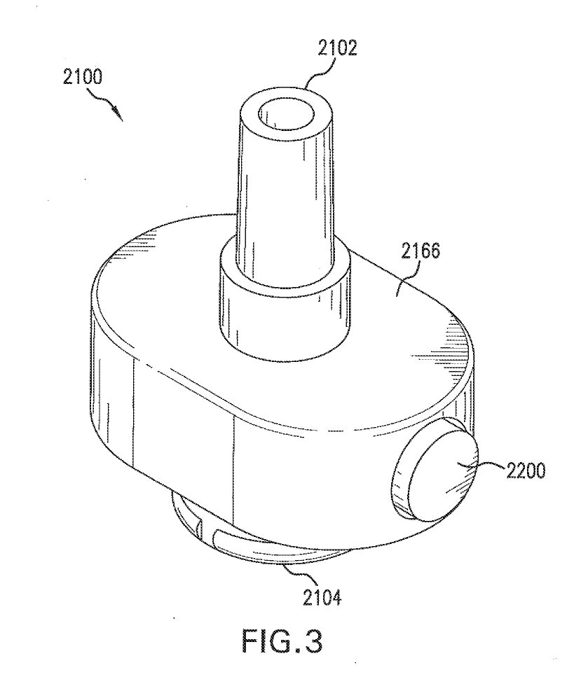

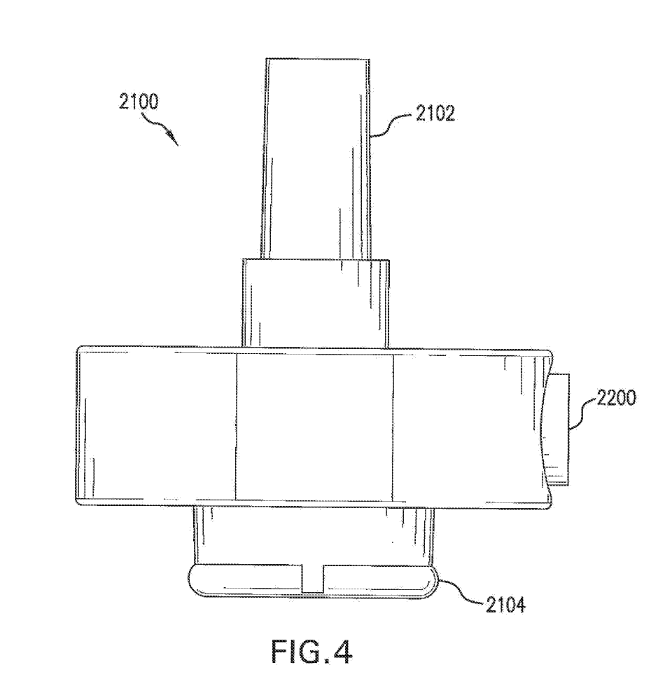

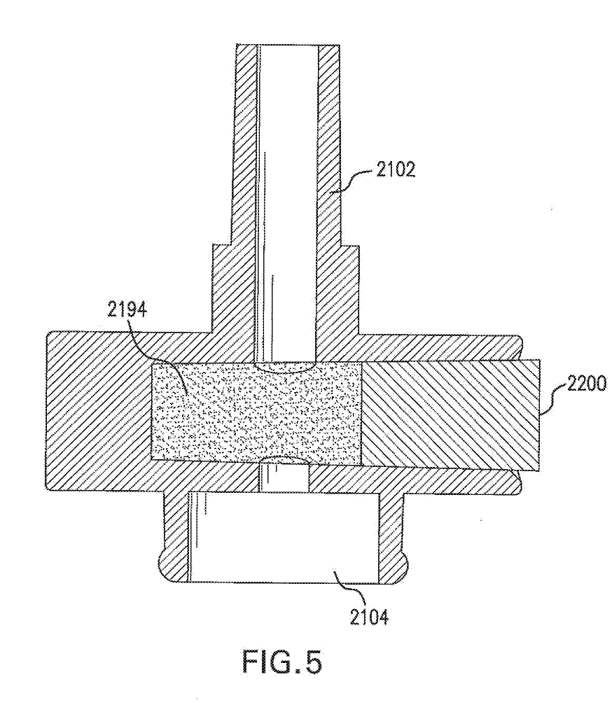

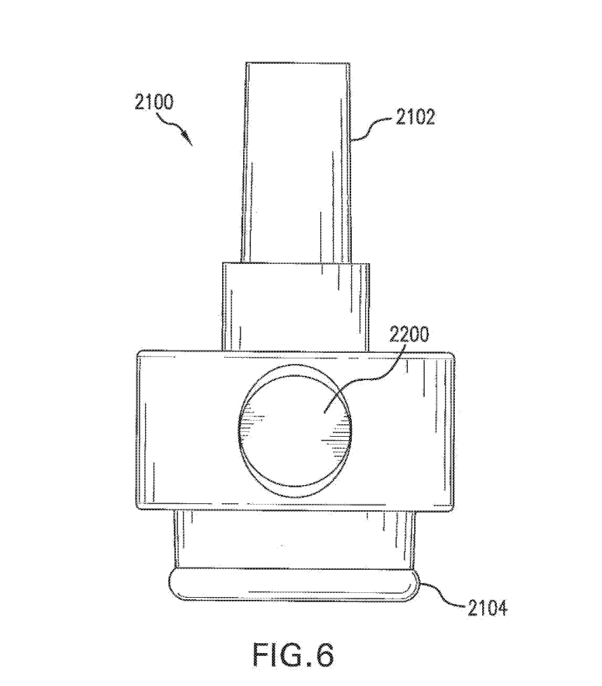

Referring to FIGS. 3-6, a second embodiment 2100 is shown, generally similar to the first described embodiment having a filter element 2194 within a filter housing 2166, the filter housing having an inlet port 2102, but including only a single sealing plug 2200 and a single outlet port 2104.

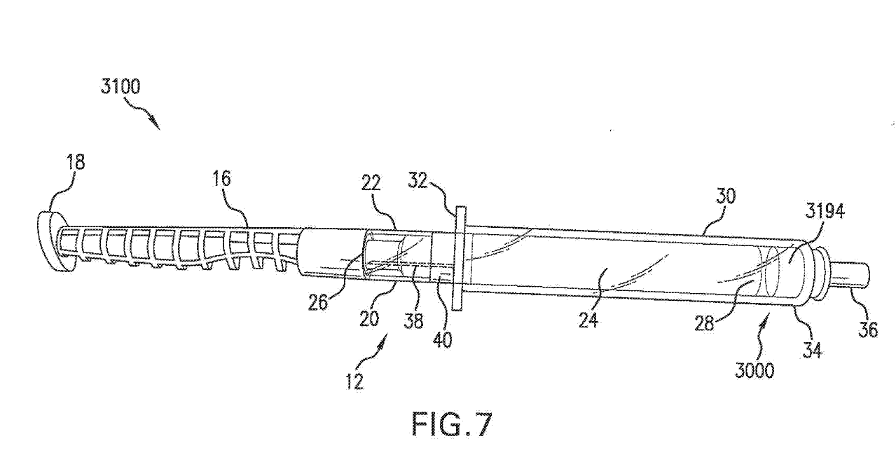

Referring to FIG. 7, a third embodiment 3100 is shown, including a sample collector 12 a described above, but further including a filter 3000 disposed within pad compression tube 30 proximate the pad compression tube second end 34. Filter 3000 includes a filter element 3194 made from a fibrous hydrophilic material which binds and filters cells and other membranous materials, DNA-containing macro structures, mucins and particulates, but which does not preferentially bind or filter RNA-containing macro structures or Total Proteins. In the embodiment, filter element 3194 consists of a glass wool plug of approximately 8 mm length. In an alternative embodiment, filter element 3194 may consist of polyolefin. In another alternative embodiment, filter element 3194 may consist of fibrous cellulose.

### Methods of Use to Obtain Rna-Enhanced, Protein-Enhanced and Dna-Enhanced Samples

Referring to FIGS. 1-2, methods to obtain RNA-enhanced, protein-enhanced, and DNA-enhanced sample are provided (in the described procedure, the sample is human saliva). Sample receivers 14a, 14b (in this case, Eppendorf tubes), are coupled to outlet ports 1104, 1106 of filter 1000. The discharge port 36 of fluid sample collector 12, forms part of pad compression tube second end 34 to express liquid sample from collection pad 24 through filter 1000 and into sample receivers 14a, 14b. Sample collector pad 24 is inserted into a patient's mouth until the sample sufficiency indicator 22 shows sufficient sample volume has been absorbed, at which point the sample collection pad 24 is inserted into the pad compression tube 30 and compressed. The liquid sample will express through sample collector discharge port/outlet 36 and thereby through filter element 1194, then distributed to sample receivers 14a, 14b, through filter discharge ports 1104, 1106.

The liquid sample deposited in sample receivers 14a and 14b will contain a substantially higher proportion of RNA and proteins to DNA, because filter element 1194 preferentially binds as well as mechanically prevents passage of large particulates, mucinous material (containing mucins, which comprise high molecular weight protein structures which coagulate or “gel” and therefore create difficulties for testing regimes directed to RNA, mRNA and proteins of interest), membranous materials such as whole cells and mitochondria containing DNA, and particulates. The polyolefin absorbent material used as a filter element preferentially binds the DNA-containing material, and mechanically filters cells, DNA-containing macrostructures (which tend to be larger than RNA-containing macrostructures) and particulate materials as well. The inventors have achieved an increase in RNA-to-DNA ratio (measured in μg RNA per μg DNA) of approximately ten-fold and higher compared to whole saliva (i.e. the RNA/DNA ratio increased by 1,000% or more). The inventors have achieved similar increases in Total Proteins-to-DNA ratio.

The RNA-enhanced and Total Protein-enhanced samples exhibit substantially improved stability at ambient temperatures, primarily due to removal of the membranous cellular materials.

An RNA stabilizer reagent may be added to the first sample volume, and a protein stabilizer may be added to the second sample volume. Alternatively, the stabilizer reagents may be pre-loaded in sample receivers 14a, 14b. Prolonged storage of samples (greater than 14 days) would still require freezing the filtered samples or some other long-term stabilization method compatible with intended downstream testing protocols.

The sample collector may then be replaced with a syringe charged with a DNA elution buffer to pass the elution buffer through filter 1000 into a separate sample receiver (not shown) to release the DNA-rich membranous material from filter element 1194 obtain a DNA-enhanced sample. The inventors have achieved an increase in DNA-to-protein ratio (measured in μg DNA/μg protein) increase of approximately five-fold and higher (i.e. the DNA/protein ratio increased by 500% or more).

The inventors have successfully used organic solvents such as isopropyl alcohol (IPA), ethanol (ETOH), and DMF (dimethylformamide) for DNA elution buffer solution, with good results. The organic solvent breaks down or weakens the membranes which contain the DNA within them, thereby releasing the DNA to flush out of filter element 1194 into a sample receiver. A different elution buffer may be preferred if IPA, ethanol or DMF would interfere with a particular downstream test regime.

Referring to FIGS. 1-2, an alternative method of providing RNA-enhanced, protein-enhanced and DNA-enhanced samples is provided, using a fluid sample distributor as described in the first embodiment 1000 (dual outlet filter), above. In the method, first and second sample receivers 14a, 14b, are coupled to outlet ports 1104 and 1106. Sample collection device pad compression tube 30 is coupled to fluid sample distributor 1000 via inlet port 1102. A fluid sample is collected from a patient as described above. The liquid specimen is expressed from sample collection pad 24 using the pad compression tube 30, as described above, passing through filter element 1194 and evenly distributed between outlet ports 1104 and 1106 into sample receivers 14a, 14b. The liquid samples in sample receivers 14a, 14b, will have enhanced RNA and protein fractions due to DNA and DNA-containing materials being preferentially retained within filter element 1194.

The same sample collector 12 and filter 1000 may then be used to provide DNA-rich samples. Sample receivers 14a, 14b, are replaced by other sample receivers (not shown) to receive DNA-rich fluid sample. Sample collection device 12 is used to draw DNA elution buffer into absorbent pad 24 by coupling or inserting pad compression tube discharge port 34 into a DNA elution buffer source, pulling back handle 16 to draw the buffer into pad compression tube 30 until a desired volume of buffer is obtained, allowing the buffer to saturate through absorbent pad 24, coupling pad compression tube discharge port 36 directly to a sample receiver, and expressing the DNA elution buffer solution from absorbent pad 24 into the sample receiver. Alternatively, the DNA elution buffer expressed from absorbent pad 24 could be flushed through filter element 1194 into a sample receiver.

In an alternative, a single outlet filter apparatus, as shown in FIGS. 3-6, may be similarly used to obtain RNA-enhanced, Total Protein-enhanced and DNA-enhanced samples.

Those skilled in the art will recognize that numerous modifications and changes may be made to the preferred embodiment without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the preferred embodiment is essential. Other embodiments are possible, their specific designs depending upon the particular application. As such, the scope of the invention should not be limited by the particular embodiments herein described but should be defined only by the appended claims and equivalents thereof.

What is claimed is:

1. An apparatus, comprising:
a. a housing, the housing including an inlet port, one or more outlet ports couplable to corresponding sample receivers, and a filter chamber; and,
b. a filter element disposed within the filter chamber, the filter element comprising a fibrous hydrophilic material which does not preferentially bind RNA, RNA-containing macrostructures or non-mucin Total Proteins.

2. The apparatus of claim 1, further comprising:
a. a sample collector having an absorbent collection pad, a pad compression tube, and a pad compression tube outlet couplable to the filter element housing inlet port; and,
b. wherein the sample collector absorbent pad is made from a material which does not differentially bind RNA, RNA-containing macrostructures or non-mucin Total Proteins.

3. The apparatus of claim 1, further comprising: wherein the filter element comprises a high-DNA affinity material.

4. The apparatus of claim 1, further comprising: wherein the filter element comprises polyolefin.

5. The apparatus of claim 4, further comprising: wherein the filter element comprises polyolefin fiber material having a density of approximate 0.077 glee.

6. The apparatus of claim 1, further comprising: wherein the filter element comprises glass wool.

7. The apparatus of claim 1, further comprising: wherein the filter element has an effective pore size in the range of 200 nm to 1000 nm.

8. The apparatus of claim 1, further comprising: wherein the filter element comprises fibrous cellulose.

9. A method, comprising the steps of:
a. obtaining a fluid sample consisting of substantially mucosal fluid;
b. expressing the fluid sample through a filter element comprising a fibrous hydrophilic material which does not differentially bind RNA, RNA-containing macrostructures or non-mucin Total Proteins; and,
c. receiving the filtered sample into one or more sample receivers for further processing.

10. The method of claim 9, wherein the step of “receiving the filtered sample into one or more sample receivers” further comprises: simultaneously receiving the filtered sample into at least two sample receivers.

11. The method of claim 9, further comprising: wherein the filter element material comprises polyolefin.

12. The method of claim 11, further comprising: wherein the filter element material has a density of approximately 0.077 g/cc.

13. The method of claim 9, further comprising: wherein the filter element material comprises glass wool.

14. The method of claim 9, further comprising: wherein the filter element material comprises cellulose.

15. The method of claim 9, further comprising the step of adding RNA stabilizer to at least one of the one or more sample receivers.

16. The method of claim 9, further comprising the step of adding protein stabilizer to at least one of the one or more sample receivers.

17. The method of claim 10, further comprising the steps of:
a. adding RNA stabilizer to a first sample receiver; and,
b. adding protein stabilizer to a second sample receiver.

18. The method of claim 9, further comprising the steps of
a. after the step of “receiving the filtered sample into one or more sample receivers for further processing”, passing a volume of DNA elution buffer through the filter element; and,
b. receiving the DNA elution buffer into at least one separate sample receiver.

19. The method of claim 10, further comprising the steps of:
a. after the step of “simultaneously receiving the filtered sample into at least two sample receivers”:
b. passing a volume of DNA elution buffer through the filter element; and,
c. receiving the DNA elution buffer into at least one separate sample receiver.

20. The method of claim 9, further comprising: wherein the filter element is contained within a filter housing having an inlet port and one or more outlet ports.

21. The method of claim 9, further comprising:
a. wherein the step of “obtaining a fluid sample consisting substantially of mucosal fluid” comprises:
b. using a sample collection device to collect a fluid sample consisting substantially of mucosal fluid, the sample collection device comprising:
i. an absorbent collection pad, a pad compression tube, and a pad compression tube discharge outlet couplable to the filter; and,
ii. wherein the sample collector absorbent pad is made from a material which does not differentially bind RNA, RNA-containing macrostructures or non-mucin Total Proteins.

22. The method of claim 20, further comprising: wherein the sample collector absorbent pad is made from fibrous polyolefin.

23. The method of claim 21, further comprising: wherein the sample collector absorbent pad material has a density of approximately 0.077 g/cc.

24. An apparatus, comprising:
a. a sample collector, the sample collector including:
i. a handle, an absorbent collection pad at least partially contained with the handle, the handle further including a sealing member;
ii. a pad compression tube insertable over the absorbent pad, handle and sealing member, the pad compression tube including at least one discharge port couplable to the filter;
b. a filter element disposed within the pad compression tube proximate the at least one discharge port, the filter element made from a material which does not preferentially bind RNA, RNA-containing macrostructures or non-mucin Total Proteins.

25. The apparatus of claim 23, further comprising: wherein the sample collector absorbent pad is made from a material which does not preferentially bind RNA, RNA-containing macrostructures or non-mucin Total Proteins.

26. The apparatus of claim 23, further comprising: wherein the sample collector absorbent pad is made from fibrous polyolefin.

27. The apparatus of claim 23, further comprising: wherein the filter element is made from fibrous polyolefin.

23. apparatus of claim 23, further comprising: wherein the filter element is made from glass wool.

29. The apparatus of claim 23, further comprising: wherein the filter element is made from fibrous cellulose.

30. The apparatus of claim 23, further comprising: wherein the pad compression tube further includes at least two discharge ports, each of the discharge ports couplable to a sample receiver to permit receiving simultaneous samples.
